(19) United States
(12) Patent Application Publication
(10) Pub. No.: US 2025/0078275 A1
(43) Pub. Date: Mar. 6, 2025

(54) MITIGATING RACIAL BIASES FOR MACHINE LEARNING BASED SKIN CANCER DETECTION

(71) Applicant: JULIAN SHAYAN ABHARI, Bixby, OK (US)

(72) Inventor: JULIAN SHAYAN ABHARI, Bixby, OK (US)

(21) Appl. No.: 18/741,697

(22) Filed: Jun. 12, 2024

Related U.S. Application Data
(60) Provisional application No. 63/472,493, filed on Jun. 12, 2023.
(60) Provisional application No. 63/538,745, filed on Sep. 15, 2023.

(51) Int. Cl. G06T 7/00

(52) U.S. Cl. CPC G06T 7/0014; G06T 2207/20081; G06T 2207/20084; G06T 2207/30088; G06T 2207/30096

(57) ABSTRACT

Machine learning based skin cancer detection tools could democratize early detection for skin cancer cases for everyone. However, due to the nature of machine learning, any communities not well represented within training data will not be able to use these tools, leading to diagnostic disparities. Available skin cancer training data represents communities of mostly first skin tones, despite skin cancer being far more lethal for people of color. The method uses domain adaptation techniques like CycleGANs to mitigate racial biases existing within machine learning based skin cancer detection tools. Domain adaptation techniques augment minority datasets, thereby improving the accuracy, precision, recall, and F1 score of typical image classification machine learning models for skin cancer classification. Finally, after creating a model and pipeline to mitigate these racial biases, the method may be integrated with mobile technology.

### CROSS REFERENCE TO RELATED APPLICATIONS

This application claims the priority of U.S. Provisional Patent Application No. 63/472,493 titled “RACIALLY UNBIASED SKIN CANCER DETECTION,” filed Jun. 12, 2023, and U.S. Provisional Patent Application No. 63/538,745 titled “MITIGATING RACIAL BIASES FOR MACHINE LEARNING BASED SKIN CANCER,” filed Sep. 15, 2023, the contents of each of which are hereby incorporated by reference.

### FIELD OF THE INVENTION

The invention relates to a method of creating a machine learning based skin cancer detector that can generalize without racial biases by creating a general model that can learn from traditional biased majority datasets and from smaller diverse datasets.

### BACKGROUND OF THE INVENTION

### 1. Introduction

Every hour two people die in the U.S. from skin cancer, according to the Skin Cancer Foundation. To make matters worse, skin cancer is far more lethal for people of color according to medical researchers. Furthermore, evidence indicates that, statistically, doctors and physicians are racially biased, leading to misdiagnoses of real cases of skin cancer. However, according to the American Academy of Dermatology, skin cancer is a disease that is 99% curable if detected early.

Artificially intelligent (AI) assisted solutions from the field of machine learning may be utilized to potentially help eliminate racial and ethnic health disparities. In the case of skin cancer, AI solutions can be particularly effective to bring early skin cancer detection tools to everyone. The application of AI to improve early detection of diseases is an example of a rare but transformative advance in medicine. The limits of this technology lie within the data. One problem is that a community's health could be severely undermined if there isn't enough data representing that community.

Unfortunately, the lack of data representing diverse racial and ethnic communities is especially apparent for skin cancer datasets.

AI skin cancer detection tools have the power to democratize early detection of skin cancer for everyone and mitigate racial and ethnic health inequalities around the world, but as the technology stands right now, AI skin cancer detection tools could potentially only worsen the health disparities due to a lack of diverse representations in the data.

Traditional data-hungry computer vision approaches are not currently ideal to mitigate racial biases, since a strong representation of data from all racial and ethnic groups is currently unavailable.

Traditional computer vision approaches for skin cancer detection use inadequate machine learning models focused on maximizing the likelihood and accuracy for the majority—not the minority—of training data. As such, using inadequate models requires gathering huge datasets to represent different demographics equally and then train these traditional machine learning models on these equally distributed demographic datasets. Due to systemic racism, this is an approach that doesn't work for skin cancer detection models since these models utilize datasets that are racially homogeneous and don't represent diverse communities. Because of these huge inequalities in diverse representations for these skin cancer datasets, traditional approaches create racial and ethnic biases that exacerbate current health disparities instead of mitigating them.

Another criticism related to bias within traditional computer vision approaches stems from how much these models actually learn. Research has shown a concerning discovery into biases within the two largest skin cancer datasets freely available. They determined that any and all machine learning models tested on these datasets will have inflated accuracies, as the models are simply picking up on spurious biases already present in the data to make their decisions, which shows further how traditional approaches can't generalize well without emerging biases.

Traditional computer vision approaches for machine learning based skin cancer detectors are prone to biases when there isn't enough data to represent every side of the problem thoroughly. Knowing that there isn't enough diverse data represented in these datasets, we must look for solutions that can make the most of the diverse data already available.

While it may seem feasible to simply gather huge diverse datasets whose scale is matched for each different demographic, these datasets simply do not exist to the same scale as the groups already represented. It is feasible, however, to gather much smaller datasets of diverse communities. A problem is how to make the most of this data and use it to generalize the models trained on much larger biased datasets without compromising accuracy.

Moreover, literature indicates that the datasets are racially biased, containing mostly lighter-skin tone data, though, according to researchers [David Wen, Saad M Khan, Antonio Ji Xu, Hussein Ibrahim, Luke Smith, Jose Caballero, Luis Zepeda, Carlos de Blas Perez, Alastair K Denniston, Xiaoxuan Liu, and et al. “Characteristics of publicly available skin cancer image datasets: A systematic review.” The Lancet Digital Health, 4(1), 2022]. Darker skin tone data exists within the 2.1% of datasets that report Fitzpatrick skin type. The problem is simply that there is a lot less data from the datasets that report skin tone. More specifically, researchers found that of the 106,950 skin lesion images that are readily available, only about 2,245 of those images have skin tone reported, this dataset being the PAD-UFES-20 dataset created from researchers [Andre G. C. Pacheco, Gustavo R. Lima, Amanda S. Salomão, Breno Krohling, Igor P. Biral, Gabriel G. de Angelo, Fábio C. R. Alves Jr, José G. M. Esgario, Alana C. Simora, Pedro B. C. Castro, and et al. “Pad-ufes-20: A skin lesion dataset composed of patient data and clinical images collected from smartphones.” Data in Brief, 32:106221, 2020]. From this dataset only about 465 of those images represent people of darker minority skin tones not usually present, Fitzpatrick types 3 through 6.

### SUMMARY OF THE INVENTION

An innovative solution is desirable to mitigate racial biases within demographics.

The disclosure teaches the use of an innovative domain adaptation technique of attention maps to isolate regions of data that contain only a mole along with style matching models to match minority data to majority data—thereby allowing a much smaller dataset to generalize.

Applicant proposes an approach that allows the use of smaller minority datasets to correct learned differences across demographic groups, creating a general model that can learn from the traditional biased datasets and smaller diverse datasets. Innovative domain adaptation technique of attention maps may be used to create a general model. The technique isolates regions of data that contain only a mole, along with utilization of style matching models to match minority data to majority data, thereby allowing for much smaller datasets to gather with effective generalizations. From the framework presented in FIG. 1 from medical researchers Agarwal, R., et al, we can clearly see that there are many ways to deal with bias.

Unlike traditional solutions, which include simply collecting more data, a novel mitigation strategy is to create a model that checks and corrects differences across demographic groups to adjust the performance of the model so that the majority group doesn't dominate the learning.

To effectively democratize early detection of skin cancer through machine learning models, it is desirable to correct learned differences across demographic groups for creating a general model that can learn from traditional biased datasets and from smaller diverse datasets. To create this general model, it is desirable to use the innovative domain adaptation technique of attention maps, which isolate regions of data that contain only a mole, along with style matching models to match minority data to majority data, which allows for much smaller datasets to gather with effective generalizations. By using style matching generative adversarial networks (GANs) for domain adaptation, minority data of cancerous and non-cancerous moles with darker skin tones may be matched to majority data of cancerous and non-cancerous moles with lighter skin tones. Furthermore, transferable attention map based methods for domain adaptation work well for mitigating learning data with different distributions in different domains. For skin cancer detection datasets, guiding a model to focus on a mole instead of on skin tone is an example of this attention map method, in which the model is focusing on the mole from these target images with mole information that connects to racially homogeneous source images. These new innovative domain adaptation techniques improve chances of creating a machine learning based skin cancer detector that can generalize without racial biases over what could be accomplished with traditional computer vision approaches.

### Overview of Racially Unbiased Computer Vision Model

1. Gather as much data of source domain-diverse images representing cancerous and non-cancerous moles of ethnicities not present in racially homogenous data sets—as possible. Identify target domain majority data for the source minority data to be mapped to—target domain is racially homogenous data.
2. Perform transferrable global attention to identify which images are better to transfer from source domain to target domain.
3. Perform transferrable local attention to specify which regions of images are better to transfer from source domain to target domain.
4. Perform style matching from source domain attention region to target domain attention region.
5. When source image is being processed, it goes through local attention network to extract the attention region and stylizes attention region to match majority data and uses a convention neural network to classify the stylized region.

The method of the invention enhances early detection of skin cancer. The method employs machine learning and domain adaptation techniques, specifically utilizing CycleGANs, to mitigate racial bias in skin cancer diagnosis. In one embodiment, the method is encapsulated in a user-friendly smartphone application. The application is aimed at democratizing access to early skin cancer detection and reducing health disparities, particularly for people of color.

The CycleGAN is a technique used for the automatic training of image-to-image translation models without paired examples. The models are trained in an unsupervised manner using a collection of images from the source and target domain that do not need to be related.

The method of the invention addresses the challenge of racial bias in medical diagnosis by leveraging machine learning and domain adaptation techniques to correct biases present in existing diagnostic tools, which often result from datasets predominantly featuring lighter skin tones. The method transforms images of minority skin types to align with majority datasets to improve diagnostic accuracy and inclusivity.

In one aspect, the disclosure relates to domain adaptation for racial inclusivity, using an individual generator component from the CycleGAN technique for image pre-processing to preserve lesion content within domain adaptation, the optimization of CycleGAN epoch selection, and a significant improvement in diagnostic metrics such as the F1 score. The technology presents a unique combination of technical features that address unmet needs in the domain of skin cancer detection. In particular, the method of the invention integrates advancements in computer vision, machine learning, and domain adaptation. The method employs an individual generator component from CycleGANs for domain adaptation and is implemented in a mobile application format, making it accessible to a broad user base.

The application operates by stylizing images of minority skin types to resemble majority datasets, thus enhancing the performance of the classification model. This results in improved accuracy, precision, recall, and F1 scores across diverse skin tones.

In greater detail, implementation includes the following steps:

The method of the disclosure is useful for implementing an unbiased computer vision model including the steps of collecting images from a real majority data set, e.g., real lighter skin tone images from real dermatologic image data, collecting images from a real minority data set, e.g., real darker skin tone images from real dermatologic image data. Creating a CycleGAN with a majority to minority generator, e.g., a light to dark skin tone generator, a minority to majority generator, e.g., a dark to light skin tone generator, and a discriminator and inputting the real majority data, e.g., the real lighter skin tone images and the real minority data, e.g., the real darker skin tone images, into the CycleGAN. Training the CycleGAN to augment the real minority data, e.g., the real darker skin tone images, to look like the real majority data, e.g., the real lighter skin tone images, and exporting the trained minority to majority generator, e.g., the dark to light skin tone generator of the CycleGAN.

Inputting images into the exported trained minority to majority generator, e.g., the exported trained dark to light skin tone generator, for outputting adapted images and classifying the adapted images.

As set forth above, the method of the disclosure may be used to facilitate representation of minority images despite a lack of data by using domain adaption techniques. A particular application is minority data being minority skin tone images and majority data being majority skin tone images. The following steps will focus on this particular application. However, it should be understood that the particular steps could be adapted for use with majority and minority sets of images of other types. Similarly, although the data and images are described as light being majority and dark being minority data, light and dark could be reversed, i.e., such that each step of Applicant's method addresses images oppositely than is described, e.g., dark to light rather than light to dark. Similarly, images and components that are described as “first” or “second” could also be reversed in an alternative method, which is hereby disclosed.

The real lighter skin tone images may be stored in a database of real first or lighter skin tone images and the real darker skin tone images may be stored in a database of real second or darker skin tone images. The database of real first or lighter skin tone images and the database of real second or darker skin tone images form a full collective image set first or lighter and second and darker, cancerous and non-cancerous image data.

The step of inputting the real first or lighter skin tone images and the real second or darker skin tone images may include cleaning the real first or lighter skin tone images to form a subset of the real first or lighter skin tone images, i.e., to form cleaned first or light CycleGAN training data 200 and cleaning the real second or darker skin tone images to form a subset of the real second or darker skin tone images, i.e., to form cleaned second or dark CycleGAN training data and inputting the cleaned subset of the real first or lighter skin tone images and the subset of real second or darker skin tone images into the CycleGAN.

As an example, the cleaned first or light CycleGAN training data and the cleaned second or dark CycleGAN training data may include two thousand total cleaned first or light skin lesion images, i.e., one thousand cleaned second or dark CycleGAN training data images and one thousand cleaned first or light CycleGAN training data images.

The cleaned first or light CycleGAN training data and the cleaned second or dark CycleGAN training data both preferably include cleaned first or light tone cancerous images, cleaned first or light tone non-cancerous images, cleaned second or dark tone cancerous images, and cleaned second or dark tone non-cancerous images.

In one embodiment, creating a CycleGAN further includes constructing the CycleGAN to comprise a second or dark to first or light skin tone generator, a first or light to second or dark skin tone generator, and a discriminator wherein the real second or darker skin tone images from the database of second or darker skin tone images are input into the second or dark to first or light skin tone generator, which outputs generated first or light images. The generated first or light images are input into the first or light to second or dark skin tone generator, which outputs reconstructed second or dark images.

The second or dark to first or light skin tone generator may be trained by inputting the reconstructed second or dark images into the second or dark to first or light skin tone generator and determining forward cycle consistency loss. The process is repeated until the forward cycle consistency loss is reduced to a desired value. Real first or lighter skin tone images from the database of first or lighter skin tone images may be input into the discriminator and the generated first or light images generated from the second or dark to first or light skin tone generator may be input into the discriminator. The discriminator makes predictions as to whether images are real or generated, for use as the discriminator loss for use as an error function to continue training. The process and predictions are repeated until the discriminator loss is reduced to a desired value.

Preferably, the real first or lighter skin tone images and the real second or darker skin tone images include identifiers that indicate skin tone information and cancerous/non-cancerous designations.

The step of inputting images into the exported trained second or dark to first or light skin tone generator for outputting adapted images may include inputting the real first or lighter skin tone images into the trained first or light skin tone generator, and inputting the real second or darker skin tone images into the trained first or light skin tone generator.

Consequently, the trained first or light skin tone generator sees only real images as inputs. The trained first or light skin tone generator outputs full adapted skin tone images including, e.g., cancerous and non-cancerous moles, including a full adapted collection of skin tone images with associated identifiers.

The full adapted collection of skin lesion images may be sorted by skin tone into categories by using the identifiers, e.g., a database may be created of clean full adapted images, e.g., the following categories:
a. a category of clean full adapted cancerous first or light skin tone images;
b. a category of clean full adapted non-cancerous first or light skin tone images;
c. a category of clean full adapted cancerous second or dark skin tone images; and
d. a category of clean full adapted non-cancerous second or dark skin tone images.

The step of classifying the adapted images may include creating an image classifier upon the full adapted collection of skin tone images in the database of clean full adapted skin tone images. The image classifier may include an adaptor cancer set including categories of full adapted cancerous images and an adapted non-cancerous set including categories of full adapted non-cancerous images.

In one embodiment, 90% full training set and a 10% full testing set is created by constructing a 90/10 split for training and testing of the cancer set or the non-cancerous set.

The classifier is, therefore, trained in a closed system by inputting only adapted images where all images have been adapted in the same way.

Cancer set images may be input into the image classifier from the 90% full training set. An example image classifier is an Inception V.3 Convolutional Neural Network 430 of (299×299×3) pixels that are from the 90% full training set. The non-cancer set images may be input into the image classifier from 90% full training set. In one embodiment, image classifier is an Inception V.3 Convolutional Neural Network 430 of (299×299×3) pixels that are from 90% full the training set. In one embodiment, the Inception V3 CNN 430 outputs (8×8×2048) tensors used for final classification.

Additionally, implementation of the generator component from the resulting CycleGAN after construction, this being Generator D to L or Generator 2 to 1, provides a unique advantage compared to other domain adaptive methods, as using the generator from the constructed CycleGAN ensures that the content of the lesion, that being the shape, the texture, and the color, is preserved after adaptation. This component allows for more information to be preserved and carried through the adaptation process, unlike other methods not using the generator component of a CycleGAN.

### BRIEF DESCRIPTION OF THE DRAWINGS

FIG. 1 shows a four-stage framework to analyze where biases can emerge.

FIG. 2 is a graphic presentation of a number of skin cancer studies by country.

FIG. 3 shows example stylized minority data to look like majority data used in domain adaptation for breast cancer data.

FIG. 4 is an example schematic of components for implementing a method of implementing a racially unbiased computer vision model of the invention.

FIG. 5 is a schematic representation of Cycle GAN training for augmenting minority dataset images to look like majority dataset images, feeding augmented images into a classifier for classification.

FIG. 6 is a schematic representation of GANs utilized to adapt data between different domains using discriminators and generators.

FIG. 7A shows example images of real benign or malignant moles of a lighter skin tone.

FIG. 7B shows example images of real benign or malignant moles of a darker skin tone.

FIG. 8A shows example images generated during Cycle GAN training, i.e., for epoch 4 of 10.

FIG. 8B shows example images generated during Cycle GAN training, i.e., for epoch 6 of 10.

FIG. 9A shows an example image of a mobile application showing a user image and a diagnosis.

FIG. 9B shows example images archived in a mobile application showing user images for various body locations.

FIG. 9C shows example images archived in a mobile application showing user images for various body locations.

FIG. 9D shows an example image of a mobile application showing a diagnostic screen providing information to a user.

FIG. 10A shows results of the Cycle GAN showing minority to majority images wherein the generator makes minority data look like majority data showing a real data sample, a generated data sample when augmented by a respective generator and a reconstructed sample showing the augmented sample passed into the generator of the original domain, i.e., augment the augmented image to appear back to its previous domain.

FIG. 10B shows results of the Cycle GAN showing majority to minority images when the generator makes majority data look like minority data showing a real data sample, a generated data sample when augmented by a respective generator and a reconstructed sample showing the augmented sample passed into the generator of the original domain, i.e., augment the augmented image to appear back to its previous domain.

FIG. 11 is a graphical representation of Cycle GAN Epochs vs CNN Accessory on three data sets.

FIG. 12 is a schematic showing an example end-user on-device unbiased processing pipeline.

FIG. 13 is a schematic showing example skin lesion shape and content preservation using a dark to light skin tone adaptor.

FIG. 14A is a schematic showing steps for implementing the method of the invention.

FIG. 14B is a schematic showing a continuation of the schematic of FIG. 14A.

FIG. 14C is a schematic showing a continuation of the schematics of FIGS. 14A and 14B.

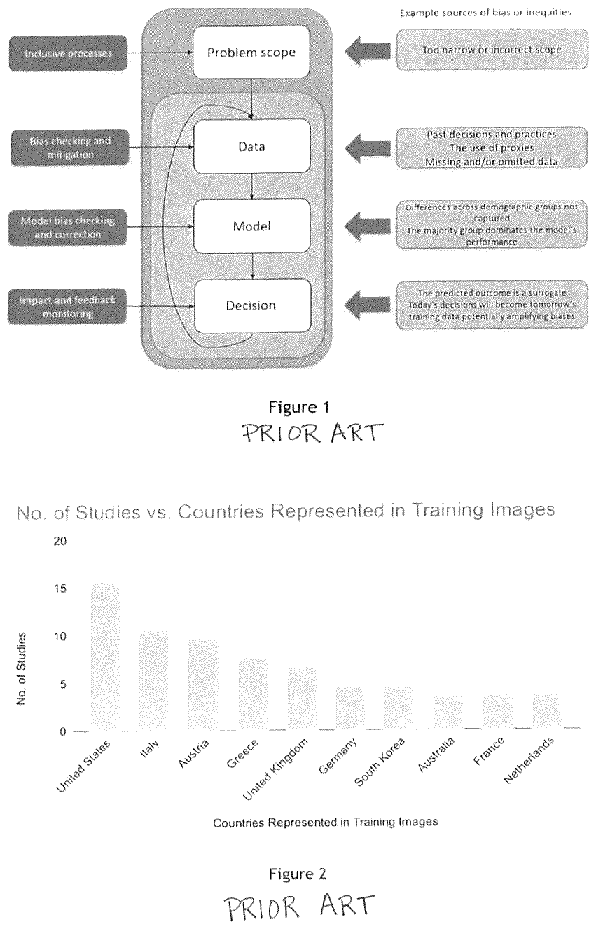

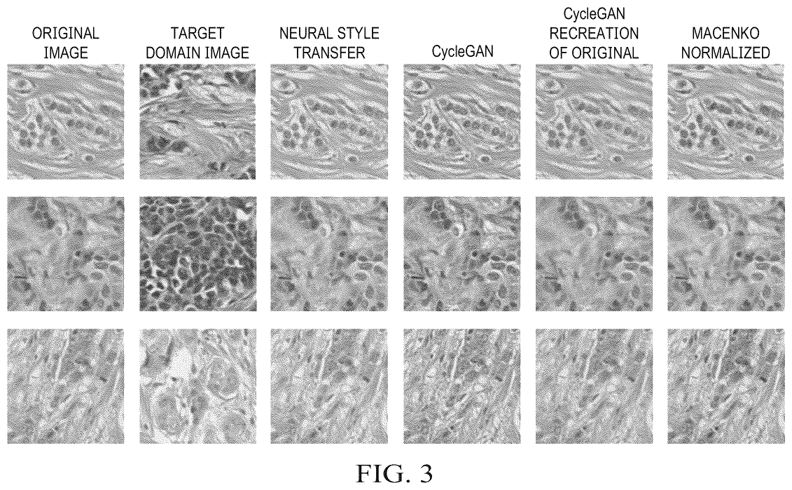

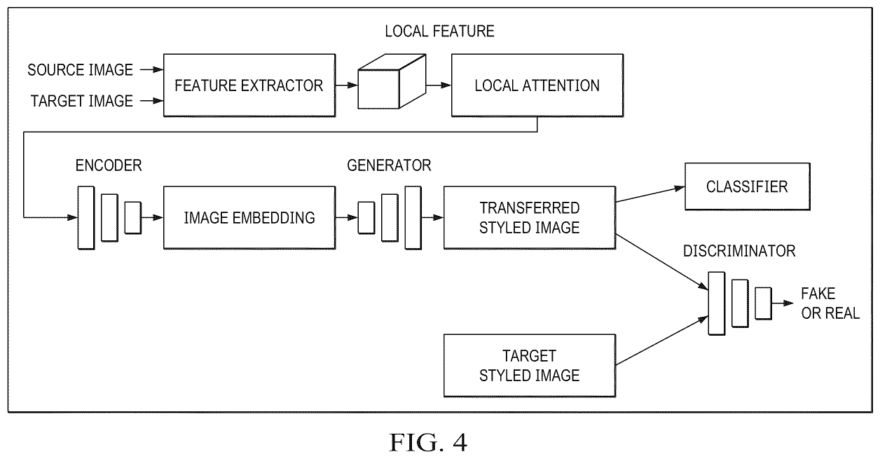

### DETAILED DESCRIPTION OF THE PREFERRED EMBODIMENTS

### 3. Methodologies

### 3.1 System Overview

Applicants' datasets play an important role in creating a classification model, as the balance of these datasets in their classes play a large factor in the resulting model's precision and recall for each classification. Although the PAD-UFES-20 dataset mentioned above contains relatively few images of people of darker minority skin tones, this example dataset can still be used to create our domain adaptation method. The PAD-UFES-20 dataset will allow for representing darker minority skin tones not present in the majority of the data, i.e., may be used as minority dataset. Real darker skin tone images 110 may be used in a database for darker skin tone images 116. An example majority dataset is the ISIC 2020 dataset. The ISIC dataset contains real lighter skin tone images placed in a database of lighter skin tone images 106.

The method of the disclosure is unique in that the method facilitates representation of minority skin tones despite a lack of data by using domain adaptation techniques. The method of the disclosure creates a typical machine learning based skin cancer detector by using deep convolutional neural networks and data from the International Skin Imaging Collaboration (ISIC) 2020 Vienna challenge. As outlined previously, datasets like the ones found from the ISIC don't represent racially or ethnically diverse communities. We should expect to see racial biases from a typical machine learning based deep convolutional neural network model trained on these racially homogeneous datasets. In one example, after training an Inception V3 architecture 430 for a deep convolutional neural network model on the ISIC 2020 Vienna challenge dataset using images of benign and malignant moles, we were able to achieve an 89.7% training accuracy with an 88.8% validation accuracy. When we tested this model on a testing dataset consisting of 592 images we omitted from the ISIC 2020 dataset we achieved 85% accuracy.

However, when tested on only the minority skin tones present in the PAD-UFES-20 dataset, or the real darker skin tone images 110 in database of darker (PAD-UFES-20 skin tone images 116, the accuracy dropped to only 50%, showing the clear bias and the need for domain adaptation.

CNN Testing Accuracy without Domain Adaption

| Majority lighter | Minority darker |
|---|---|
| skin tone data | skin tone data |
| 85% | 50% |

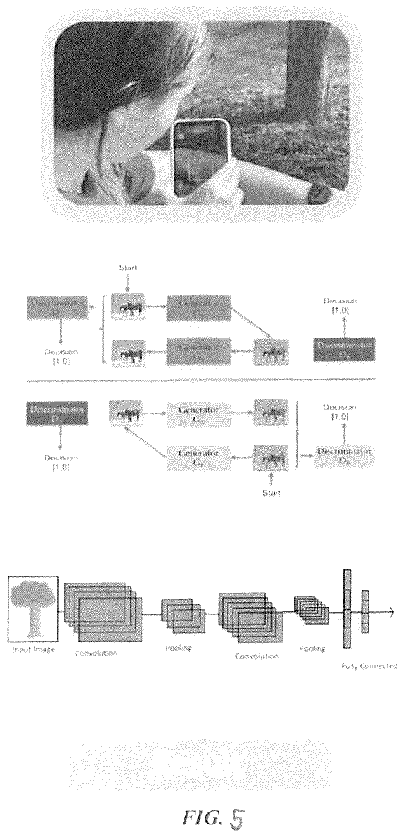

A CycleGAN was created to adapt our minority darker skin tone data or the real darker skin tone images 110 in database of darker skin tone images 116, to our majority lighter skin tone data or real lighter skin tone images 100 placed in database of lighter skin tone images 106. After training CycleGAN 250 for 5 to 10 epochs for adapting the PAD-UFES-20 minority dataset, i.e., to adapt the database of darker real skin tone images 116, to look like the majority ISIC 2020 dataset, i.e., the database of lighter skin tone images 106, and for outputting generated light images 290. With these adaptations to our full dataset collection, we created an Inception V3 architecture 430 for a Conventional Neural Network, i.e., a full image classifier using the IV3 architecture, 400 and 430 and trained that on the adapted minority data with the majority data, i.e., database of lighter skin tone images 106. Finally, after creating a classification model, i.e., image classifier 400, trained on the full collection of adapted images 312 we tested image classifier 400 on adapted images created using CycleGAN 250 for darker skin tones 320c, 320d from a cleaned adapted set 320 of the full collection of adapted images 312, i.e., adapting the PAD-UFES-20 dataset with the CycleGAN Generator_{(D to L)}. When used in a mobile application, the data-pipeline process starts with a captured photo that is then augmented to look like the majority dataset, i.e., to look like the real lighter skin tone images 100 in database of lighter skin tone images 106, using CycleGAN 250. The augmented photo is then fed into classifier 400 that was trained and tested on similarly augmented minority and majority data, resulting in a classification. See, e.g., FIG. 5.

### Domain Adaptation

Generative adversarial networks (GAN) are used in domain adaptation techniques, as they can alter data from one domain to match data of another domain. In the method of the disclosure, CycleGAN 250 is used, which was created by researchers Jun-Yan Zhu et al., see, e.g., Jun-Yan Zhu, Taesung Park, Phillip Isola, and Alexei A. Efros 2017, “Unpaired image-to-image translation using cycle-consistent adversarial networks.” 2017 IEEE International Conference on Computer Vision (ICCV).

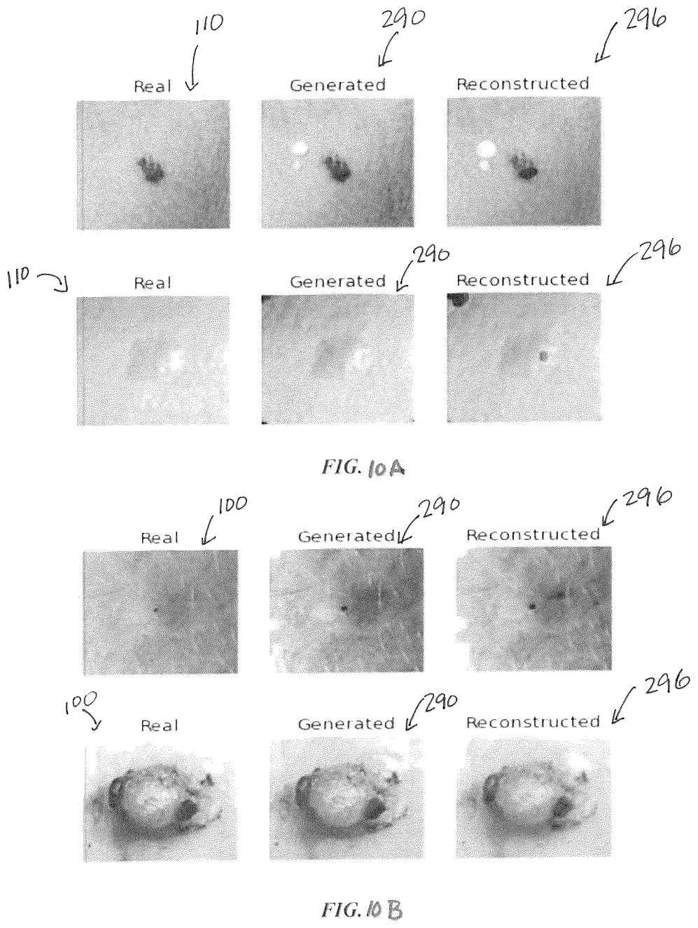

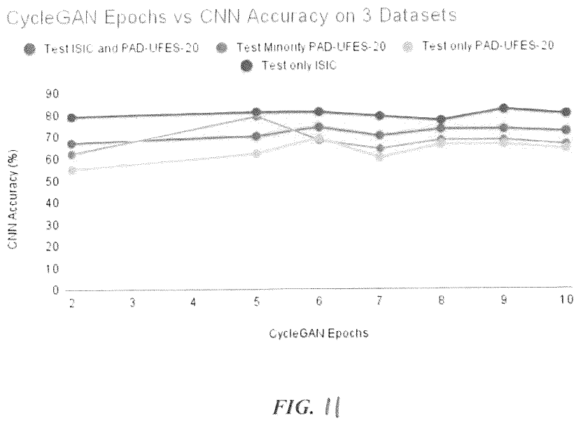

The CycleGAN 250 (see, e.g., FIG. 5) is a specific type of generative adversarial network that uses multiple loss functions to ensure cycle consistency when adapting data between different domains, and ensures that the content of the data is preserved. More specifically, CycleGAN architecture uses a first discriminator to adapt to the target domain and uses a second discriminator for training the CycleGAN in reverse. The two discriminators provide the first two loss functions, ensuring that the generated images could exist in the corresponding domain. Then as an image from domain L, e.g., real lighter skin tone images 100, is passed to generator L_to_D, it will create a generated image, e.g., generated light image 290, as shown in FIG. 10B, by stylizing the generated image to approximate domain D. Then that generated image is passed to generator L, e.g., generator 260, to create a reconstituted image to stylize it back to domain L. The difference between the original image and the reconstructed dark image 290 through the forward cycle process determines the forward cycle consistency loss. The same process can be done in reverse domains to determine backward cycle consistency loss.

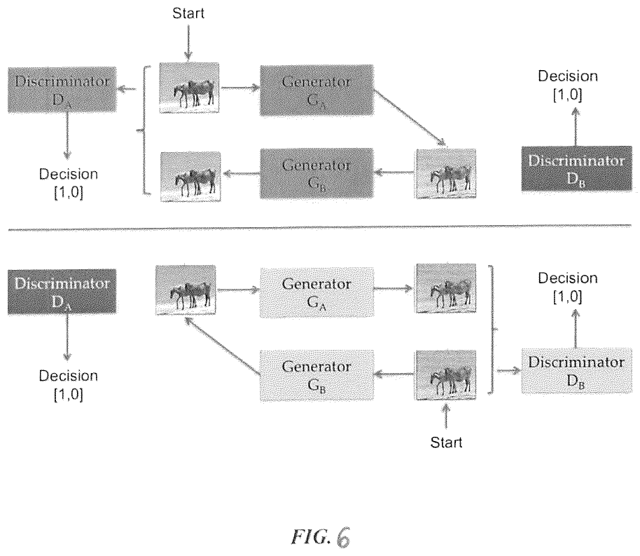

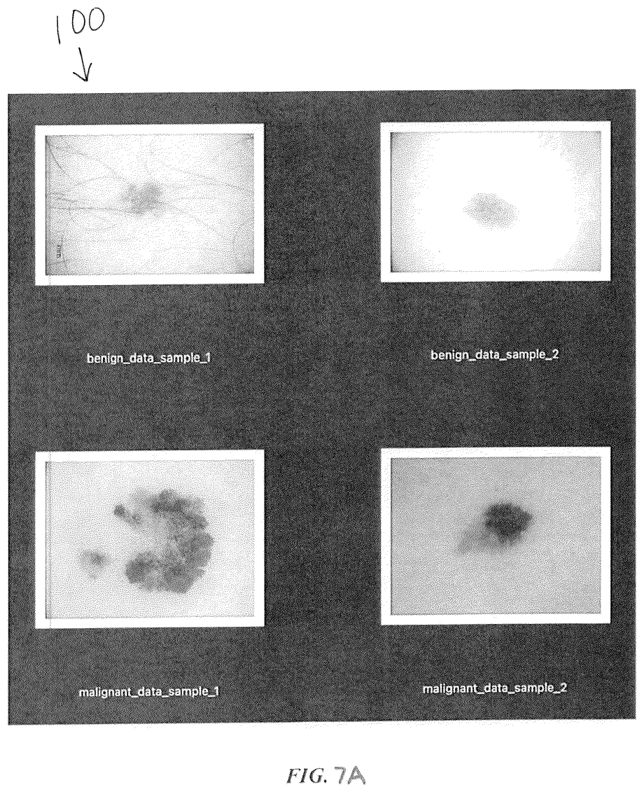

The same process can be used in a special case wherein identity consistency loss function is determined by having an input image already from the target domain be passed into the generator to that specific domain, e.g., a real lighter skin tone image is passes into the dark to light generator, which should approximate it to belong in the target domain. However, since the image is initially of that generator's target domain, there should be no change when the image is passed to that generator, e.g., a real lighter skin tone image when generated to belong to Domain L should have no change. The evaluation of the input image from the target domain and the augmented version when passed through the generator whose target domain is the input image's domain determines the identity loss. See FIG. 6, which comes from [Amita Kapoor. “Hands-on Artificial Intelligence for IoT.” 2019 Packt Publishing].

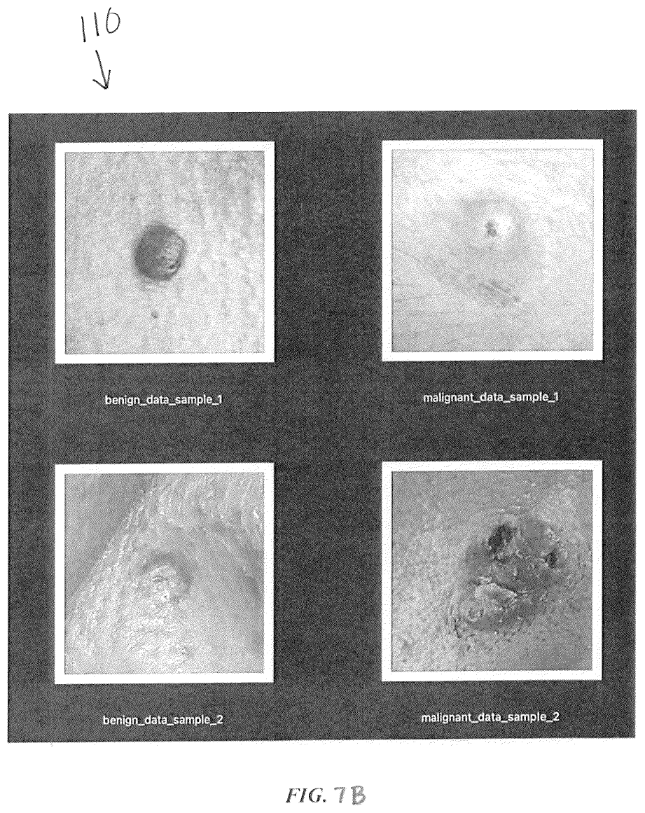

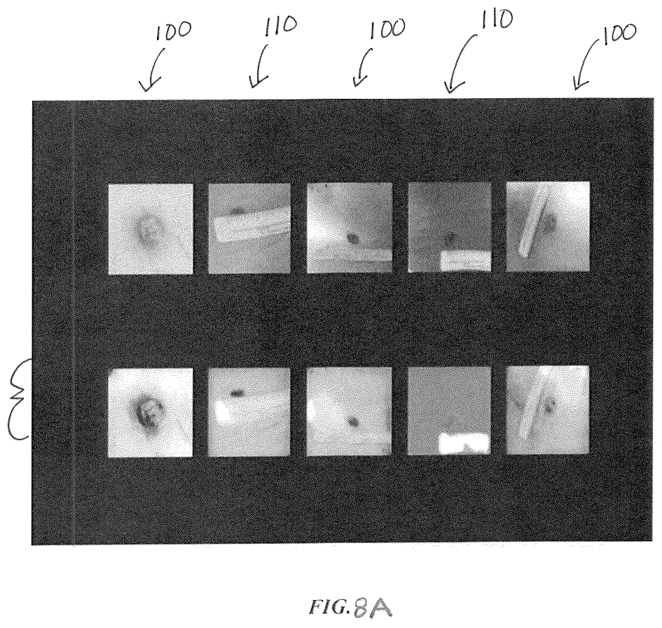

Applicants' majority dataset of real lighter skin-tone images 106 will be referred to as domain L. A minority dataset of real darker skin-tone images 116 will be referred to as domain D. When given an image of a benign or malignant mole of a darker skin tone, domain D, the generator for domain L, e.g., generator 260, should be able to make the image look like it was of a lighter skin tone, as shown in FIGS. 7A and 7B.

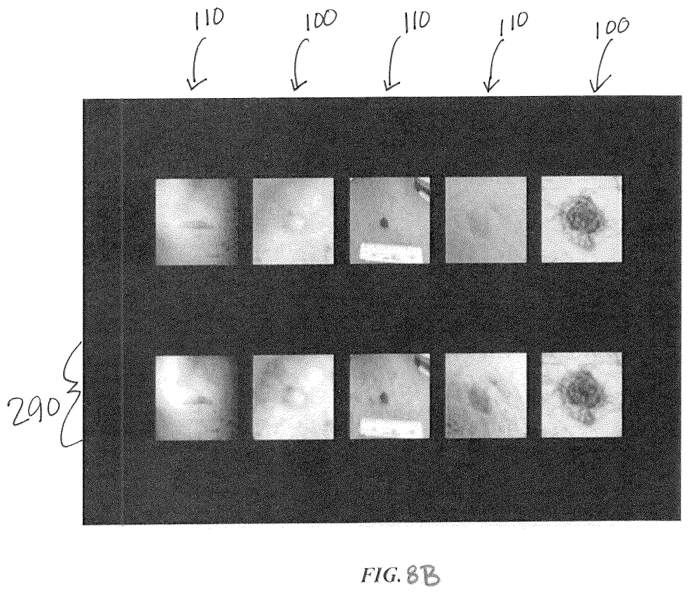

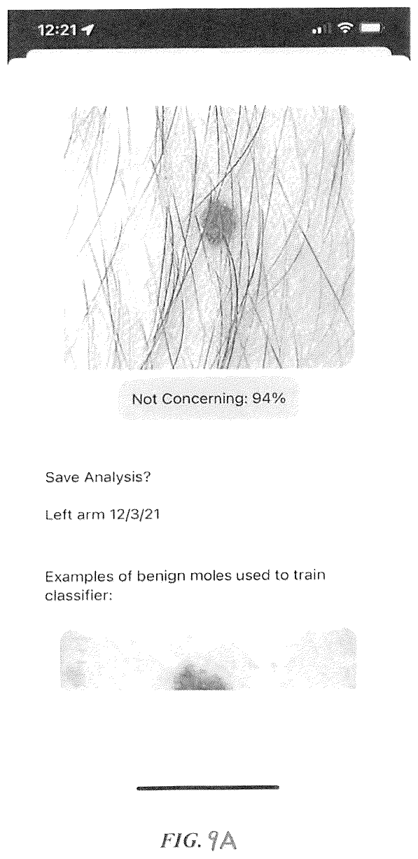

The architecture of this technique requires a lot of computing power to train, which can serve as a limitation for training this model. However, as soon as the model is finished training, the generator of a specific domain can be exported as a file to fit a mobile application, e.g., as adapter generator 300. We trained our CycleGAN using the majority and minority skin cancer datasets as previously mentioned. The CycleGAN has trained for 10 epochs. We saved an instance of both generator models after each epoch. The results of training for the minority to majority domain generator for epoch four and six respectively, are shown in FIGS. 8A and 8B. In these figures the real samples, i.e., from real darker skin tone images 100, are shown on top, with the generator's adaptation, shown beneath. Quickly, the generator learns to adapt the skin tone difference. However, artifacts may be created by the model through the process. As can be seen in the Figures, i.e., FIGS. 8A and 8B, in epoch four there are clear gaps in the image, shown as empty spots of white or black pixels. By epoch six these artifacts appear to be a lot less apparent, however.

### Mobile System

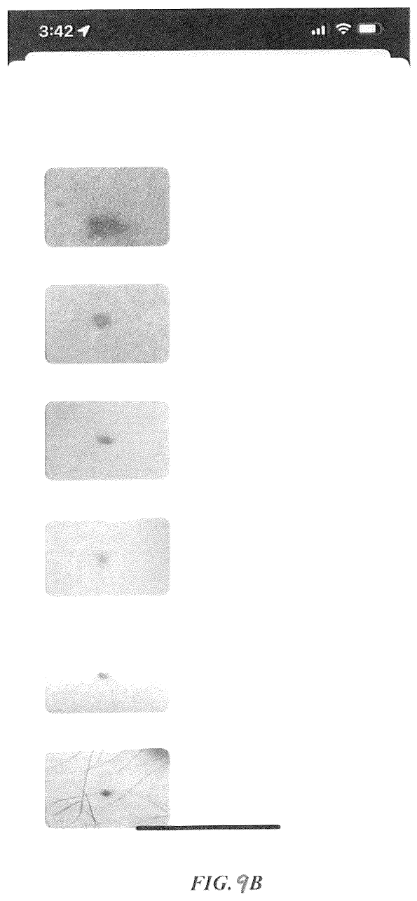

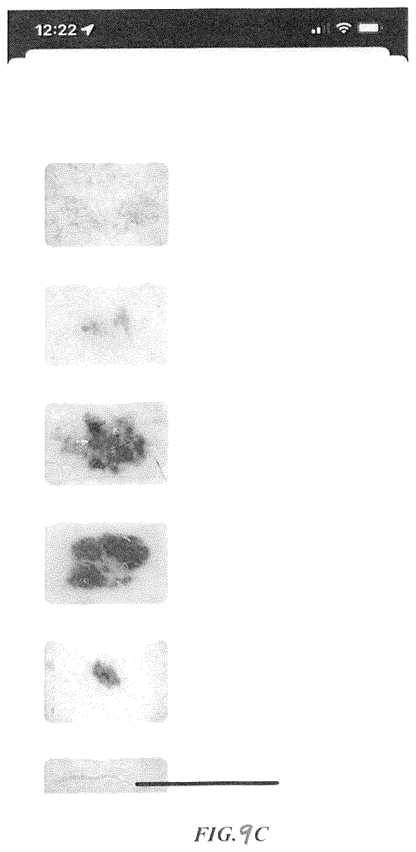

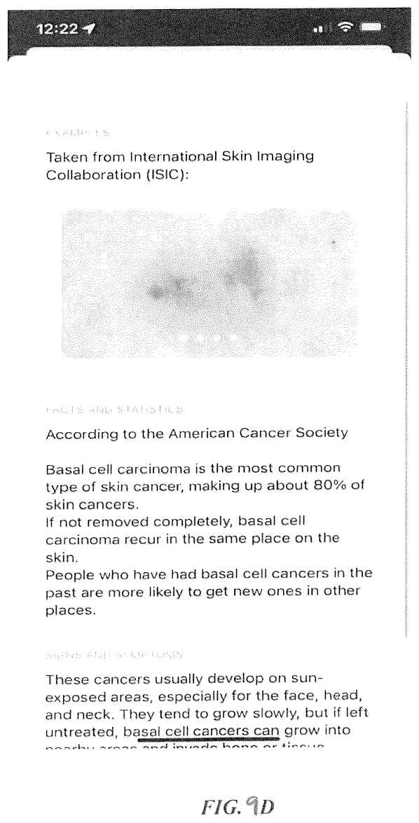

A mobile application designed to be used on iOS devices was also developed to use the model pipeline previously outlined to provide assessments regarding how similar a mole resembled skin cancer (see, e.g., FIGS. 9A-9D). With this capacity to provide an informed and individual recommendation, the app made a modest contribution to solving the problem of equal access to early skin cancer detection. Features were developed to allow a user to track their mole over time, as cancers such as melanoma can develop as quick as 6-weeks. Therefore an archive that allowed users to track changes of their moles was created. Furthermore, an index was provided of common skin cancer types showed images, facts and statistics, and a location on their body to monitor for each skin cancer type to help educate a user. See example images in FIGS. 9A-9D.

Implementing the classification pipeline required the use of CoreML libraries to export a tensorflow model, the minority to majority generator from the CycleGAN, as a CoreML model file extension to be used by iOS devices for augmenting user captured photos. Furthermore, exporting the Inception V3 CNN classification model was done in a similar manner using CoreML libraries. With the CoreML files the models were easily implemented by using the iOS device's camera to output an image fed into the generator. The generator's output was converted into an image to be fed into the classification model.

### Results

The results of the CycleGAN 250 are described and shown in two different figures, i.e. FIGS. 10A and 10B, which show minority to majority generated images 290 (FIG. 10A) and majority to minority generated images (FIG. 10B). The generator, e.g., generator 260, 300 to make the minority data look like the majority is shown with great results, as the skin lesion and its contents barely change, yet the darker skin tone of the minority image is changed to appear lighter, as we hoped for.

The generator to make majority lighter tone skin lesions look like minority darker skin tone images can also be shown with great results. Again the skin lesion itself rarely changes, yet the skin tone becomes visibly darker. FIGS. 10A and 10B show a real data sample, what the data sample looks like when augmented by the respective generator, and show the reconstructed sample, which is what happens when the augmented sample is passed into the generator of the original domain, which should augment the augmented image to appear back into its previous domain.

When training and testing the classification model on the majority data and augmented minority data we were able to achieve a jump from 50% accuracy for the minority datasets without Cycle-GAN augmentation to 79% accuracy with a 0.71 F1 score for the benign classification and a 0.83 F1 score for the malignant classification, when tested on the minority dataset augmented using the CycleGAN. This shows our success in domain adaptation.

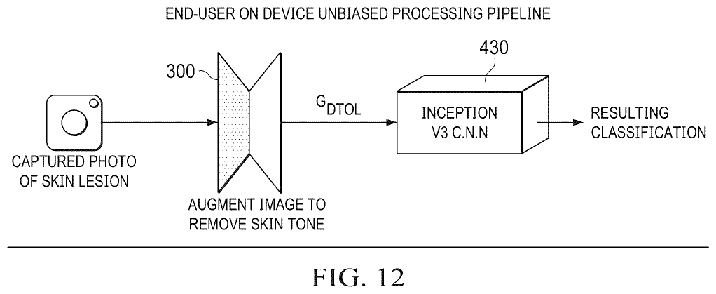

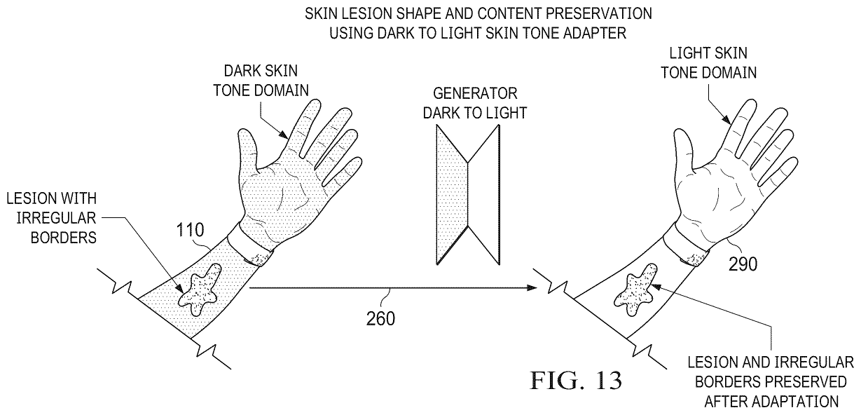

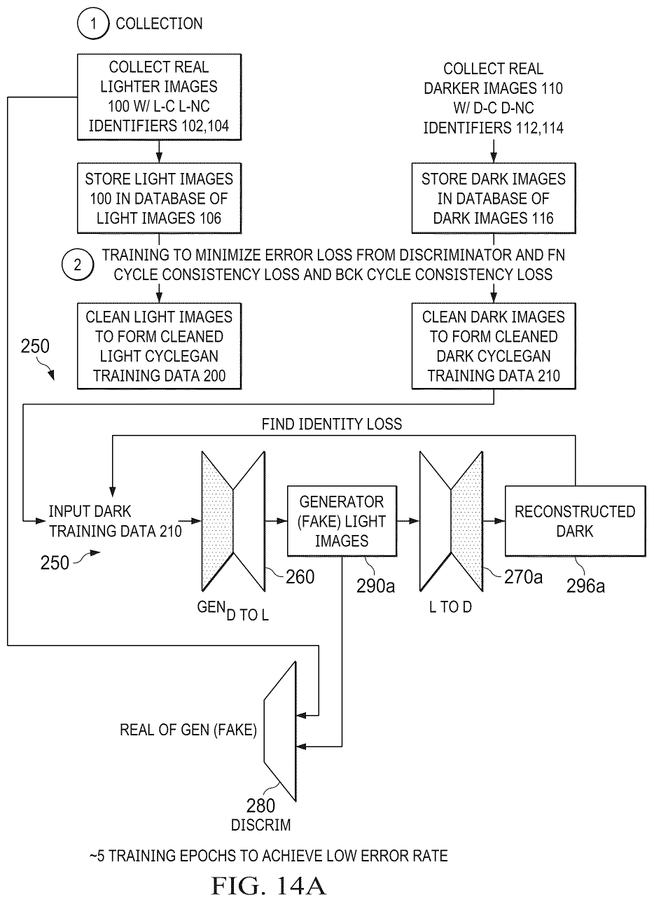

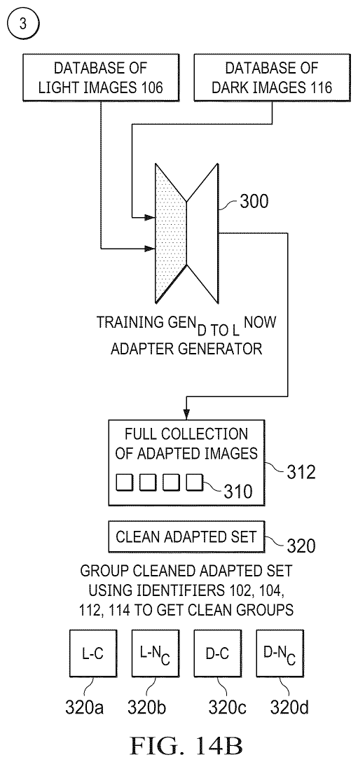

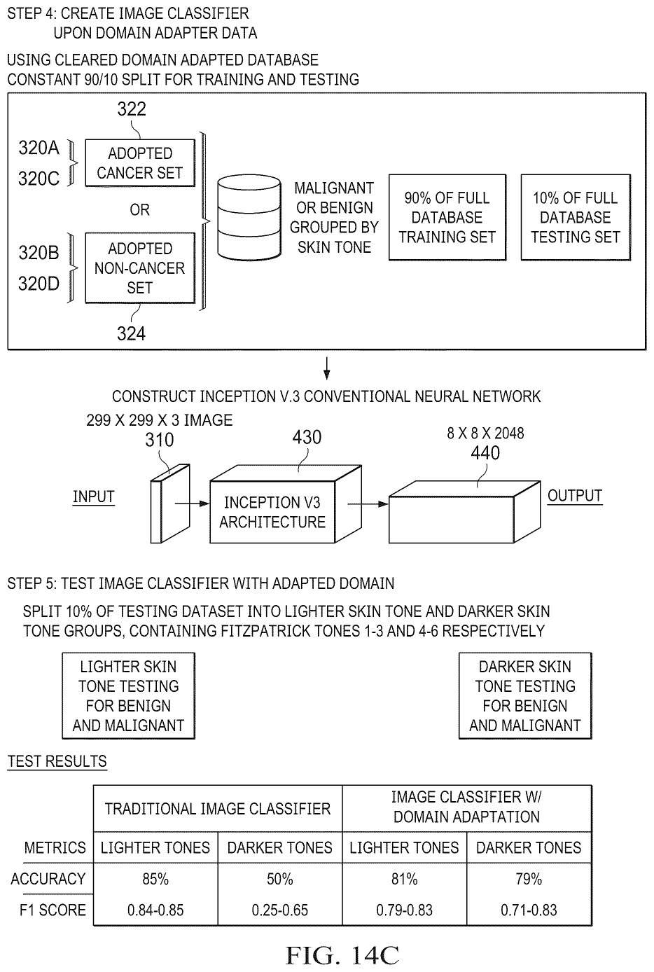

Interestingly, the amount of epochs that the CycleGAN was trained varied the results when tested using the classification model. More specifically, after training the CycleGAN model for 10 epochs, individual instances of the CycleGAN trained at different epochs were used to create multiple variations of augmented minority data, which was tested to train and test the classification model. What we found was that the 5th epoch CycleGAN resulted in the smallest gap between classification accuracy for minority darker skin tone images and majority lighter skin tone images. The classification model trained and tested on minority data that was augmented with the 5th epoch CycleGAN resulted in our best results of 79% accuracy for minority darker skin tones and 81% accuracy for majority lighter skin tones, yielding the smallest gap between accuracies that we achieved. See FIG. 11 for a graph showing CycleGAN Epochs v CNN Accuracies on three Datasets.

### Discussion

Applicants' methodology was a great success when using a small dataset of minority darker skin tone images for domain adaptation, however, using this process with larger datasets of minority darker skin tone images are expected to yield better results and more robust tests.

### CONCLUSION

The goal was to create a machine learning based skin cancer detector that would be able to have as good of an F1 score and accuracy for darker skin tone images as lighter skin tone images, thereby mitigating the racial biases learned in machine learning based skin cancer detection. By using a CycleGAN to augment the minority data and correct the learned differences between the domains, we were able to train and test a classification model that achieved 79% accuracy, with a 0.71 F1 score for benign classifications, and a 0.83 F1 score for malignant classifications when tested on minority darker skin tone data, thereby mitigating racial biases for machine learning based skin cancer detection.

In greater detail, the method of the invention includes the steps of collecting real skin tone images. In particular, real lighter skin tone images 100 are collected from real dermatologic image data. An example of real dermatologic data are images uploaded to the International Skin Imaging Collaboration (ISIC). Each of real lighter skin tone images 100 is provided with cancerous identifier information and non-cancerous identifier information.

Real light skin tone images 100 are stored in a database of lighter skin tone images 106 that comprises a Domain L.

Real darker skin tone images 110 are also collected from real dermatologic image data. An example of real dermatologic image data for collecting real darker skin tone images 110 are images downloaded from PAD-UFES-20 dataset accessible at Mendeley data. Each of these images are correlated with cancerous identifier information 112 and non-cancerous identifier information 114.

Real darker skin tone images 110 are stored in a database of darker skin tone images 116 that comprises a Domain D. The database of lighter skin tone images 106 and the database of darker skin tone images 116 form a full collective image set, i.e., a total database of lighter and darker, cancerous and non-cancerous image data 120, which comprises a Domain of Total Data.

### Step 2

CycleGAN 250 is created with CycleGAN skin tone generators 260 and 270.

### Part A

Cleaning said total database of lighter and darker, cancerous and non-cancerous image data 120 to form cleaned light CycleGAN training data 200 and a clean dark CycleCAN training data 210. An example of cleaning includes removal of confusing images, out of focus images, images taken at bad angles, and the re-sizing of images. As an example, 1000 total cleaned light skin lesion images make up the cleaned light CycleGAN training data 200 and 1000 cleaned dark images make up the cleaned dark CycleGAN training data 210.

Cleaned light CycleGAN training data 200 and cleaned dark CycleGAN training data 210 together comprise cleaned light tone cancerous images, cleaned light tone non-cancerous images, cleaned dark tone cancerous images, and cleaned dark tone non-cancerous images.

### Part B

CycleGAN 250 is constructed comprising CycleGAN Generator_{(DtoL)} 206 and CycleGAN Generator_{(LtoD)} 270.

Real darker skin tone images 110 from database of darker skin tone images 116 are input into CycleGAN Generator_{(DtoL)} 260, which outputs generally light images 290. Additionally, generated light images 290 are input into CycleGAN Generator_{(LtoD)} 270, which outputs reconstructed dark images 296. CycleGAN 205 is trained by inputting reconstructed dark images 296 into Generator_{(DtoL)} 260 and determining forward cycle consistency loss. The training process is repeated until the forward cycle consistency loss is reduced to a desired value.

CycleGAN 250 may be run in reverse and trained by inputting reconstructed light images 250 into Generator_{(LtoD)} 206 and determining backwards cycle consistency loss. The training process is repeated until the backward cycle consistency loss is reduced to a desired value.

### Next Step

Inputting real lighter skin tone images 100 from database of lighter skin tone images 106 into CycleGAN discriminator 280 and inputting generated light images 290 into discriminator 280 wherein discriminator 280 attempts to predict whether each image is real or generated. The resulting prediction is used as discriminator loss, which is used as an error function to continue training. The discriminator process is repeated until a desired rate of failure to discriminate between real images, i.e., real images 100, 110, and generated images, i.e., generated light images 290 are continued until the discriminator loss is reduced to a desired value.

### Step 3

The real lighter skin tone images 100 and real darker skin tone images 110 are preprocessed to create an adapted domain of data.

First, the total database of lighter and darker cancerous and non-cancerous image data 120 is adapted using now trained Generator_{(DtoL)} 260. Trained Generator_{(DtoL)} 260 shall henceforth be referred to as adapter generator 300.

First real lighter skin tone images 100 in the database of lighter skin tone images 106 are input into adapter generator 300.

Real darker skin tone images 110 in the database of darker skin tone images 112 are input into adapter generator 300.

Adapter generator 300, therefore, sees only real images 100, 110 that retain identifier information 102, 104, 112, 114 as inputs. Identifiers 102, 112 include skin tone information, e.g., a Fitzpatrick type data while identifiers 104, 114 relate to a cancerous/non-cancerous designation. Adapter generator 300 outputs full adapted skin tone images, including cancerous and non-cancerous moles comprising a full adapted collection of skin tone images 312.

Full adapted collection of skin tone images 312 are sorted by skin tone into categories by using identifier information 102, 104, 112, 114 to create a database of full clean adapted images 320. The database of full clean adapted images 320 includes the following categories of images: (1) a category of clean full adapted cancerous light skin tone images 320a; (2) a category of clean full adapted non-cancerous light skin tone images 320b; (3) a category of clean full adapted cancerous dark skin tone images 320c; (4) a category of clean full adapted non-cancerous dark skin tone images 320d.

### Step 4—Classifier

An image classifier 400 is created upon the full adapted collection of skin tone images 312. In the database of clean full adapted skin tone images 320. The database of clean full adapted skin tone images 320 includes an adapted cancer set 322, comprised of the categories of full adapted cancerous images 320a and 320c. The database of clean full adapted skin tone images 320 additionally includes an adapted non-cancerous set 324 comprised of the categories of full adapted non-cancerous images 320b and 320d.

A 90% adapted training set 410 is created and a 10% adapted testing set 420 is created by constructing a 90/10 split for training and testing of the adapted cancerous set 322 or the adapted non-cancerous set 324.

Classifier 400 is therefore trained in a closed system by inputting only adapted images from the 90% of the adapted training set 410 where all images have been adapted in the same way, i.e., the input is comprised of the adapted cancer set 322 or the adapted non-cancer set 324 of images. In a preferred embodiment, an inception V.3 Convolutional Neural Network 430 is used in the classifier 400.

In one embodiment, the inception V.3 Convolutional Neural Network 430 outputs (8×8×2048) tensors 440 used for final classification.

### Step 5—Testing

Image classifier 400 is tested with 10% adapted testing set 420.

10% adapted testing set 420 is split into lighter skin tones testing, for example, comprising images with Fitzpatrick tones 1-3. The 10% adapted testing set 420 includes cancerous and non-cancerous images. The splitting of the testing set may be achieved by utilizing identifiers 102, 104. The 10% adapted testing set 420 is also split into a darker skin tone testing set 510, for example, into a set comprising images showing Fitzpatrick tones 4-6. Darker skin tone testing set 510 includes cancerous and non-cancerous images. The splitting of the adapted testing set into lighter skin tone testing set 500 and darker skin tone testing set 510 may be achieved utilizing identifiers 102, 104, 112, 114.

In one embodiment, a traditional image classifier may be run, as is known in the art, on the database of lighter skin tone images 102 and on the database of darker skin tone images 112 for generating traditional prediction results 520 using a 90/10 split of training and testing. Traditional prediction results 520 comprise an accuracy score, an F1 score for lighter tones, and comprises and accuracy score and an F1 score for darker tones as shown in the below table.

Improved prediction results 530 are generated from light skin tone testing set 500 and dark skin tone testing set 510. Improved prediction results 530 comprise and accuracy score and an F1 score for lighter tones and comprise an accuracy score and an F1 score for darker tones. A comparison of the traditional prediction results 520 and the improved prediction results 530 show better performance for dark tones, as shown in the below table.

TABLE 1

| | | | CNN with 5- | CNN with 5- |
|---|---|---|---|---|
| | Traditional | Traditional | Epoch | Epoch |
| | Image | Image | CycleGAN | CycleGAN |
| | Classifier | Classifier | (ours) | (ours) |
| | Lighter | Darker | Lighter | Darker |
| Metrics | Tones | Tones | Tones | Tones |
| Accuracy | 85% | 50% | 81% | 79% |
| F1 score | 0.84-0.85 | 0.25-0.63 | 0.79-0.83 | 0.71-0.83 |

The method of the disclosure may be achieved by various combinations of the steps outlined in the following numbered paragraphs.

1. A method of the disclosure for implementing an unbiased computer vision model comprises the steps of using a trained small component of CycleGAN Generator, e.g., (Generator_D_to_L) or Generator 2 to 1 in a full data processing pipeline, which facilitates the use of domain adaptation and makes the most out of small datasets, yielding high accuracy for, even limited, minority datasets.

2. A method of the disclosure according to paragraph 1 for implementing an unbiased computer vision model comprises the steps of collecting real first or lighter skin tone images 100 from real dermatologic image data, collecting real second or darker skin tone images 110 from real dermatologic image data, creating a CycleGAN with a second or dark to first or light skin tone generator, a first or light to second or dark skin tone generator, and a discriminator, inputting the real first or lighter skin tone images and the real second or darker skin tone images into the CycleGAN, training the CycleGAN to augment the real second or darker skin tone images to look like the real first or lighter skin tone images, exporting the trained second or dark to first or light skin tone generator of the CycleGAN, inputting images into the exported trained second or dark to first or light skin tone generator for outputting adapted images, classifying the adapted images.

3. The method according to any of paragraphs 1 and 2 wherein the real first or lighter skin tone images are stored in a database of real first or lighter skin tone images 106, wherein the real second or darker skin tone images are stored in a database of real second or darker skin tone images 116, wherein the database of real first or lighter skin tone images 106 and the database of real second or darker skin tone images 116 form a full collective image set first or lighter and second or darker, cancerous and non-cancerous image data 120.

4. The method according to any of paragraphs 1-3 wherein the step of inputting the real first or lighter skin tone images and the real second or darker skin tone images further comprising the steps of cleaning the real first or lighter skin tone images to form a subset of the real first or lighter skin tone images, i.e., to form cleaned first or light CycleGAN training data 200, cleaning the real second or darker skin tone images to form a subset of the real second or darker skin tone images, i.e., to form cleaned second or dark CycleGAN training data 210, inputting the cleaned subset of the real first or lighter skin tone images and the subset of real second or darker skin tone images into the CycleGAN.

5. The method according to any of paragraphs 1-4 wherein the cleaned first or light CycleGAN training data and the cleaned second or dark CycleGAN training data comprise two thousand total cleaned first or light skin lesion images.

6. The method according to any of paragraphs 1-5 wherein the cleaned first or light CycleGAN training data 200 and the cleaned second or dark CycleGAN training data 210 both comprise cleaned first or light tone cancerous images, cleaned first or light tone non-cancerous images, cleaned second or dark tone cancerous images, and cleaned second or dark tone non-cancerous images.

7. The method according to any of paragraphs 1-6 wherein the step of creating a CycleGAN further comprises constructing the CycleGAN 250 comprising the second or dark to first or light skin tone generator 260, a first or light to second or dark skin tone generator 270, and the discriminator 280, inputting the real second or darker skin tone images 110 from the database of second or darker skin tone images 112 into the second or dark to first or light skin tone generator 260, which outputs generated first or light images 290, inputting the generated first or light images 290 into the first or light to second or dark skin tone generator 270, which outputs reconstructed second or dark images 296.

8. The method according to any of paragraphs 1-7 further comprising, training the second or dark to first or light skin tone generator by inputting the reconstructed second or dark images 296 into the second or dark to first or light skin tone generator and determining forward cycle consistency loss, and repeating until the forward cycle consistency loss is reduced to a desired value.

9. The method according to any of paragraphs 1-8 further comprising:
inputting the real first or lighter skin tone images 100 from the database of first or lighter skin tone images 102 into the discriminator 280, inputting the generated first or light images 290 generated from the second or dark to first or light skin tone generator into the discriminator 280, wherein the discriminator 280 makes predictions as to whether images are real or generated, for use as the discriminator loss for use as an error function to continue training, repeating the predictions until the discriminator loss is reduced to a desired value.

10. The method according to any of paragraphs 1-9 wherein the real first or lighter skin tone images 100 include identifiers 102, 104, wherein the real second or darker skin tone images 110 include identifiers 112, 114, and wherein the step of inputting images into the exported trained second or dark to first or light skin tone generator for outputting adapted images comprises inputting the real first or lighter skin tone images 100 into the trained first or light skin tone generator 300, inputting the real second or darker skin tone images 110 into the trained first or light skin tone generator 300, wherein the trained first or light skin tone generator 300 sees only real images 100, 102 as inputs, wherein the trained first or light skin tone generator outputs full adapted skin tone images including cancerous and non-cancerous moles 310 comprising a full adapted collection of skin tone images 312 with associated identifiers.

11. The method according to any of paragraphs 1-10 further comprising a step of sorting the full adapted collection of skin lesion images 312 by skin tone into categories by using the identifiers 102, 104, 112, and 114, creating a database of clean full adapted images 320, the categories comprising:
a. a category of clean full adapted cancerous first or light skin tone images 320a;
b. a category of clean full adapted non-cancerous first or light skin tone images 320b;
c. a category of clean full adapted cancerous second or dark skin tone images 320c; and
d. a category of clean full adapted non-cancerous second or dark skin tone images 320d.

12. The method according to any of paragraphs 1-11 wherein the step of classifying the adapted images comprise creating an image classifier 400 upon the full adapted collection of skin tone images 312 in the database of clean full adapted skin tone images 320 comprising an adaptor cancer set 322 comprised of categories of full adapted cancerous images 320a and 320c and an adapted non-cancerous set 324 comprised of categories of full adapted non-cancerous images 320b and 320d, and creating a 90% full training set 410 and a 10% full testing set 420 by constructing a 90/10 split for training and testing of the cancer set 322 or the non-cancerous set 324, training the classifier 400 in a closed system by inputting only adapted images where all images have been adapted in the same way.

13. The method according to any of paragraphs 1-12 further comprises inputting the cancer set 322 images into the image classifier 400 from 90% full training set 410.

14. The method according to any of paragraphs 1-13 wherein the image classifier is an Inception V.3 Convolutional Neural Network 430 of (299×299×3) pixels that are from 90% full training set 410.

15. The method according to any of paragraphs 1-14 comprises inputting the non-cancer set 324 images into the image classifier 400 from 90% full training set 410.

16. The method according to any of paragraphs 1-15 wherein the image classifier is an Inception V.3 Convolutional Neural Network 430 of (299×299×3) pixels that are from 90% full training set 410.

17. The method according to any of paragraphs 1-16 wherein the Inception V3 CNN 430 outputs (8×8×2048) tensors 440 used for final classification.

Thus, the present invention is well adapted to carry out the objectives and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the spirit of this invention as defined by the claims.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A method of implementing an unbiased computer vision model comprising the steps of:
collecting real first skin tone images from real dermatologic image data;
collecting real second skin tone images from real dermatologic image data;
creating a CycleGAN with a second to first skin tone generator, a first to second skin tone generator, and a discriminator;
inputting said real first skin tone images and said real second skin tone images into said CycleGAN;
training said CycleGAN to augment said real second skin tone images to look like said real first skin tone images;
exporting said trained second to first skin tone generator of said CycleGAN;
inputting images into said exported trained second to first skin tone generator for outputting adapted images;
classifying said adapted images.

2. The method according to claim 1 wherein:
said real first skin tone images are real lighter skin tone images;
said real second skin tone images are real darker skin tone images;
said first to second skin tone generator is a light to dark skin tone generator; and
said second to first skin tone generator is a dark to light skin tone generator.

3. The method according to claim 1 wherein:
said real first skin tone images are stored in a database of real first skin tone images;
said real second skin tone images are stored in a database of real second skin tone images;
said database of real first skin tone images and said database of real second skin tone images form a full collective image set first and second, cancerous and non-cancerous image data.

4. The method according to claim 1 wherein said step of inputting said real first skin tone images and said real second skin tone images further comprising the steps of:
cleaning said real first skin tone images to form a subset of said real first skin tone images, i.e., to form cleaned first CycleGAN training data;
cleaning said real second skin tone images to form a subset of said real second skin tone images, i.e., to form cleaned second CycleGAN training data;
inputting said cleaned first Cycle GAN training data and said cleaned second CycleGAN training data into said CycleGAN.

5. The method according to claim 4 wherein:
said cleaned first CycleGAN training data and said cleaned second CycleGAN training data comprise two thousand total cleaned first skin lesion images.

6. The method according to claim 4 wherein:
said cleaned first CycleGAN training data and said cleaned second CycleGAN training data both comprise cleaned first tone cancerous images, cleaned first tone non-cancerous images, cleaned second tone cancerous images, and cleaned second tone non-cancerous images.

7. The method according to claim 1 wherein said step of creating a CycleGAN further comprises:
constructing said CycleGAN comprising said second to first skin tone generator, a first to second skin tone generator, and said discriminator;
inputting said real second skin tone images into said second to first skin tone generator, which outputs generated first images;
inputting said generated first images into said first to second skin tone generator, which outputs reconstructed second images.

8. The method according to claim 7 further comprising:
training said second to first skin tone generator by inputting said reconstructed second images into said second to first skin tone generator and determining forward cycle consistency loss;
repeating until said forward cycle consistency loss is reduced to a desired value.

9. The method according to claim 7 further comprising:
inputting said real first skin tone images into said discriminator;
inputting said generated first images generated from said second to first skin tone generator into said discriminator, wherein said discriminator makes predictions as to whether images are real or generated, for use as the discriminator loss for use as an error function to continue training;
repeating said predictions until said discriminator loss is reduced to a desired value.

10. The method according to claim 1 wherein:
said real first skin tone images include identifiers;
said real second skin tone images include identifiers; and wherein said step of inputting images into said exported trained second to first skin tone generator for outputting adapted images comprises:
inputting said real first skin tone images into said trained first skin tone generator;
inputting said real second skin tone images into said trained first skin tone generator;
wherein said trained first skin tone generator sees only real images as inputs;
wherein said trained first skin tone generator outputs full adapted skin tone images including cancerous and non-cancerous moles comprising a full adapted collection of skin tone images with associated identifiers.

11. The method according to claim 10 further comprising a step of:
sorting said full adapted collection of skin lesion images by skin tone into categories by using said identifiers;
creating a database of clean full adapted images, said categories comprising:
a. a category of clean full adapted cancerous first skin tone images;
b. a category of clean full adapted non-cancerous first skin tone images;
c. a category of clean full adapted cancerous second skin tone images; and
d. a category of clean full adapted non-cancerous second skin tone images.

12. The method according to claim 11 wherein said step of classifying said adapted images comprise:
creating an image classifier upon said full adapted collection of skin tone images in said database of clean full adapted skin tone images comprising an adaptor cancer set comprised of categories of full adapted cancerous images and an adapted non-cancerous set comprised of categories of full adapted non-cancerous images;
creating a 90% full training set and a 10% full testing set by constructing a 90/10 split for training and testing of said cancer set or said non-cancerous set;
training said classifier in a closed system by inputting only adapted images where all images have been adapted in the same way.

13. The method according to claim 12 further comprises:
inputting said cancer set images into said image classifier from 90% full training set.

14. The method according to claim 13 wherein:
said image classifier is an Inception V.3 Convolutional Neural Network of (299×299×3) pixels that are from 90% full training set.

15. The method according to claim 12 comprises:
inputting said non-cancer set images into said image classifier from 90% full training set.

16. The method according to claim 13 wherein:
said image classifier is an Inception V.3 Convolutional Neural Network of (299×299×3) pixels that are from 90% full training set.

17. The method according to claim 14 wherein:
said Inception V3 CNN outputs (8×8×2048) tensors used for final classification.
